(19) United States
(12) Patent Application Publication
(10) Pub. No.: US 2024/0335621 A1
(43) Pub. Date: Oct. 10, 2024

(54) CLOSURE CAP AND CLOSURE SYSTEM FOR A SYRINGE THAT IS PREFILLED WITH A MEDICAMENT OR MEDICAL ACTIVE SUBSTANCE

(71) Applicant: INDUCTIO AG, Solothurn (CH)

(72) Inventor: Horst KOLLER, Uznach (CH)

(73) Assignee: INDUCTIO AG, Solothurn (CH)

(21) Appl. No.: 18/579,119

(22) PCT Filed: Jul. 14, 2022
(86) PCT No.: PCT/EP2022/069815

§ 371 Date: Jan. 12, 2024

(30) Foreign Application Priority Data
Jul. 14, 2021 (EP) 21185683.6

(51) Int. Cl. A61M 5/34

(52) U.S. Cl. CPC A61M 5/347

(57) ABSTRACT

A closure cap (2, 2′) for a syringe body (40, 40′) prefilled with a medicament or medical active substance is intended to reliably ensure a high degree of leak-tightness with simple handling. For this purpose, the closure cap (2, 2′) according to the invention comprises a rigid carrier cap (6, 6′) provided with an external thread (18) in a contact region (16), which has an inner channel (14) extending in a longitudinal direction, in which a sealing plug (8, 8′) made of a softer and more deformable material than the carrier cap (6, 6′), preferably made of rubber or TPE, is arranged.

The invention relates to a closure cap for a medication container, in particular for a syringe that is prefilled with a medicament or medical active substance. It further relates to a closure system comprising such a closure cap.

Medication, especially for treatment in highly specialized or complex therapies, is usually provided in active substance or medication containers, also known as containers or vials. Alternatively, however, medications can also be provided in prefilled syringes or syringe bodys. Such prefilled syringes, which usually make subsequent handling by staff much easier, particularly when administering the active substance, usually comprise the actual syringe body filled with the active substance, to which the syringe needle is attached, for example screwed on, for the purpose of administration immediately before the syringe is used.

Modern medical procedures or therapies in particular can involve the use of drugs or substances that are actually toxic or otherwise harmful or dangerous. This can pose acute and long-term health risks to staff handling such substances, such as pharmacists and nurses, especially if they are repeatedly exposed to drugs or solvents that could escape into the air during the preparation, administration of drugs and other similar treatments. This problem can be particularly serious when cytotoxins, antiviral drugs, antibiotics or radiopharmaceuticals are involved. The potential health risks arising from exposure to these medicaments include an increased risk of cancer, genetic changes and the like. Furthermore, in view of the fact that drugs with an extremely high dose price have recently been approved, it is urgently desirable or even necessary to reliably prevent the unintentional release of even the smallest quantities of such medicaments or active substances into the environment.

It is therefore generally desirable to design the systems or components used in the administration of medications for particularly low material or active substance losses during the entire handling chain. For containers in which the medication is held for a short time or over a certain period of time, i.e. in particular for prefilled syringes or prefilled syringe bodys, it is therefore desirable for the closure system to be highly leak-proof. In addition, the components used should of course also be particularly easy and reliable to handle and also cost-effective.

The invention is therefore based on the task of providing a closure cap for a medicine container, in particular for a syringe prefilled with a medicament or medical active substance or a syringe body prefilled with a medicament or medical active substance, with which a high level of tightness can be reliably achieved with simple handling. It further relates to a closure system comprising such a closure cap.

With regard to the closure cap, this task is solved according to the invention with a rigid carrier cap which is provided with an external thread in a contact area for screwing onto the mouth area of the syringe and which has an internal channel extending in a longitudinal direction in which a sealing plug made of a material which is softer and more deformable than the carrier cap, preferably made of rubber or TPE, is arranged. In particular, the inner channel provided in the carrier cap for receiving the sealing plug is provided with a circumferential latching edge. Correspondingly, according to one aspect of the invention, the sealing plug has a peripheral latching edge adapted to it on the outside. In particular, this is dimensioned and positioned in such a way that the sealing plug, after being inserted into the inner channel, is fixed in the inner channel of the carrier cap by interlocking the two latching edges.

Advantageous embodiments of the invention are the subject of the subclaims.

The invention is based on the consideration that the desired high level of tightness when closing the prefilled syringe body can be achieved by providing a sealing element whose material properties are specifically designed for this sealing effect.

This should consist of a suitable material with suitable plastic properties that is compatible with the base materials normally used for such syringe bodys, such as glass or plastic in particular. However, in order to ensure adequate protection against external influences and thus a high level of reliability during temporary storage of the active substances, this sealing element should be surrounded on the outside by a carrier cap designed in the manner of a protective cap. The closure cap can thus be designed in the form of a two-component design, whereby the desired high sealing effect is provided by the sealing element suitably designed in this respect in the form of a functional separation and, on the other hand, the mechanical stability and resilience is provided by the protective carrier cap.

The design of the closure cap is therefore basically two-component. For easier handling, however, the components should preferably be pre-assembled, so that only one component has to be applied when the closure cap is actually attached to the syringe body. Accordingly, according to one aspect of the invention, the inner stopper provided as a sealing element is fixed in the inner channel of the carrier cap in a snap-in manner.

In this embodiment, the carrier cap can also be designed for particularly easy and therefore user-friendly assembly on the syringe body by being provided with an external thread at a suitable position for connection to the syringe body.

With regard to the choice of material, the components mentioned are advantageously designed specifically for the intended use for medical active substances and compatibility with other materials commonly used in this field. For this purpose, the carrier cap is advantageously made of a plastic, preferably polypropylene (PP), a polyolefin, cyclo-olefin copolymer (COC), cyclo-olefin polymer (COP) or polycarbonate. In a further advantageous embodiment, both the sealing plug and the carrier cap are each made of “medical grade” material.

In order to make handling particularly easy, the closure cap is designed for attachment to and subsequent removal from the syringe body by screwing, i.e. by screwing on or off; this is achieved in particular by the external thread provided on the carrier cap. In order to make the screwing or unscrewing process particularly easy for the operating personnel, the carrier cap is provided in a particularly advantageous design with suitable gripping elements which make it particularly easy for the operator to apply force during the screwing movement. For this purpose, in a particularly advantageous embodiment, the support cap is provided on its outer circumference with a number of gripping grooves, preferably two, extending in the longitudinal direction, in a handling area that is offset from the contact area when viewed in the longitudinal direction.

The external thread of the carrier cap is preferably designed as a Luer thread.

In an alternative embodiment, which is considered to be inventive in its own right, the closure cap can also be produced by a two-component injection molding process in which the two components are produced together from the outset.

The closure cap described above with its preferred embodiments is basically intended to be screwed onto the actual syringe body. It can therefore be used directly if the syringe body is already equipped with a suitable thread system according to its basic design, for example in order to be able to screw a needle holder or the like onto the mouth area of the syringe body at a later stage.

In another design, syringe bodies of the type mentioned can be provided with a tapered mouth area onto which, for example, the needle holder can be clamped when using the syringe. In order to be able to use the closure cap also with such a syringe body, also referred to as a “standard syringe”, a closure system for a syringe prefilled with a medicament or medical active substance with a closure cap of the type mentioned is provided in an embodiment which is regarded as independently inventive. In addition to said closure cap, the closure system also comprises a threaded adapter piece which is provided in an outer jacket with an internal thread adapted to the external thread of the carrier cap and which has a clamping ring which can be pushed onto an end region of the syringe body and is provided with a number of circumferentially arranged clamping springs. Advantageously, the threaded adapter piece is made of a plastic, preferably polypropylene (PP), a polyolefin, cyclo-olefin copolymer (COC), cyclo-olefin polymer (COP) or polycarbonate.

The outer cap forms a tamper-evident closure for the closure cap in a particularly preferred embodiment that is considered to be inventive in its own right. For this purpose, the receiving basket can, for example, be provided with latching elements which latch with the closure cap when it is inserted, so that removal is no longer possible. For access to the active substance, i.e. to “open” and prepare the syringe sealed in this way, a lid area of the outer cap can then be torn off through suitable predetermined breaking points in the outer cap, for example, so that the closure cap is exposed and thus made accessible.

The outer cap can be adapted to the outer diameter of the syringe by a suitable choice of geometry and size of its outer sheath in an embodiment which is also regarded as independently inventive, so that when the outer cap is fitted, a continuous outer sheath is formed for the syringe, consisting of its outer sheath and the outer sheath of the outer cap adapted thereto. In this sense, by suitably varying the parameters, a similar closure system—while retaining the “inner” components, namely the sealing stopper and the carrier cap—can also be used for syringe bodies of different dimensions; the outer cap thus serves to adapt the outer contours in this sense. In this way, a considerably increased usability of the elements can be achieved, especially for particularly large production quantities.

The advantages achieved with the invention consist in particular in the fact that the multi-component design of the closure cap means that a majority of actually competing design objectives, namely, for example, high tightness, high reliability, mechanical resilience and/or good manageability, can be achieved in equal measure. In addition, an intuitively usable twist-off mechanism is provided, in particular via the suitable contouring of the carrier cap in its outer area. The closure cap ensures the integrity of the container or syringe body, but can still be opened easily. It also provides a seamless fit for pre-filled glass or polymer syringes. Container closure integrity is generally an important concern in the pharmaceutical industry. This is achieved in the same way as user-friendliness, which is also important.

An embodiment of the invention is explained in more detail with reference to a drawing. It shows:

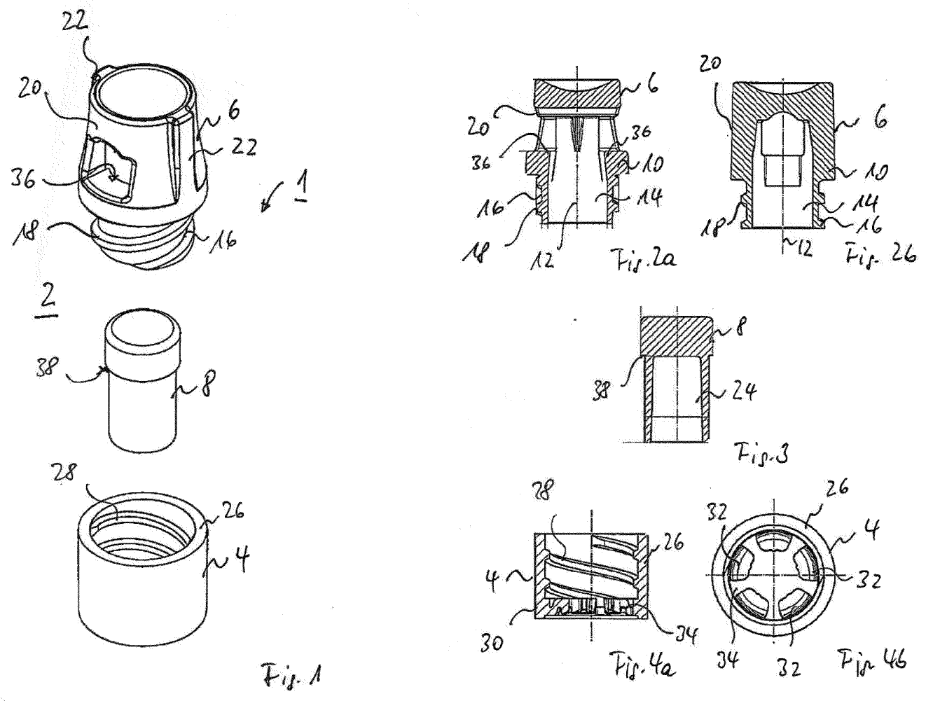

FIG. 1 An exploded view of a closure system for a medicine container, in particular for a syringe prefilled with a medicament or medical active substance,

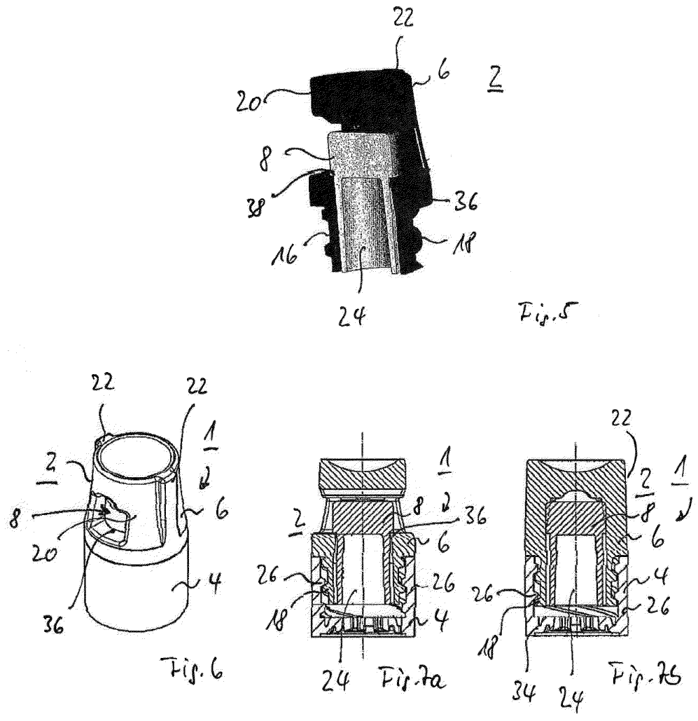

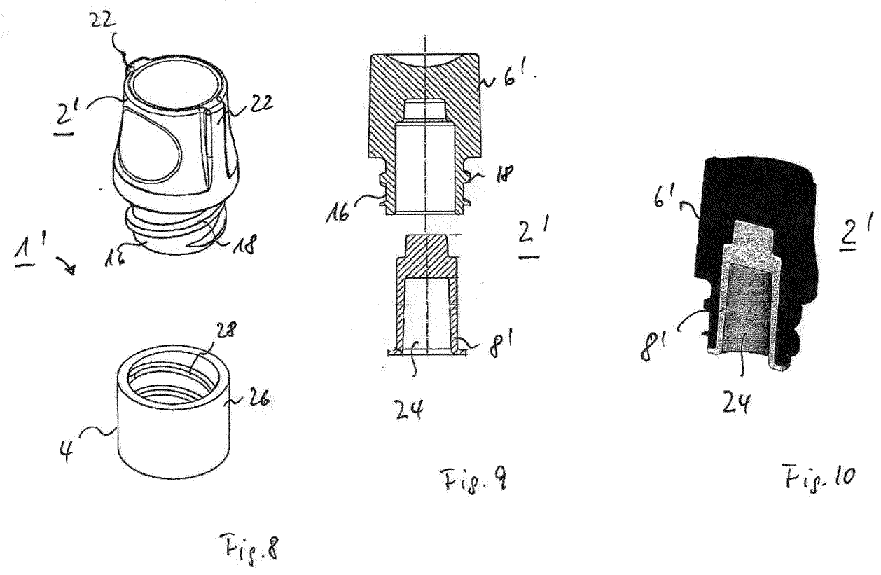

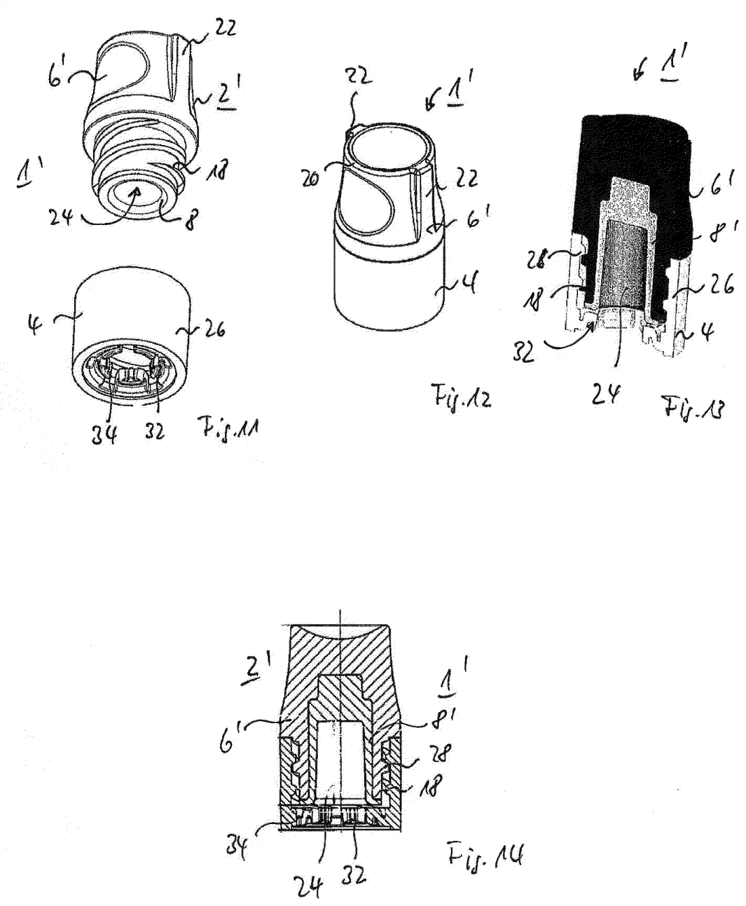

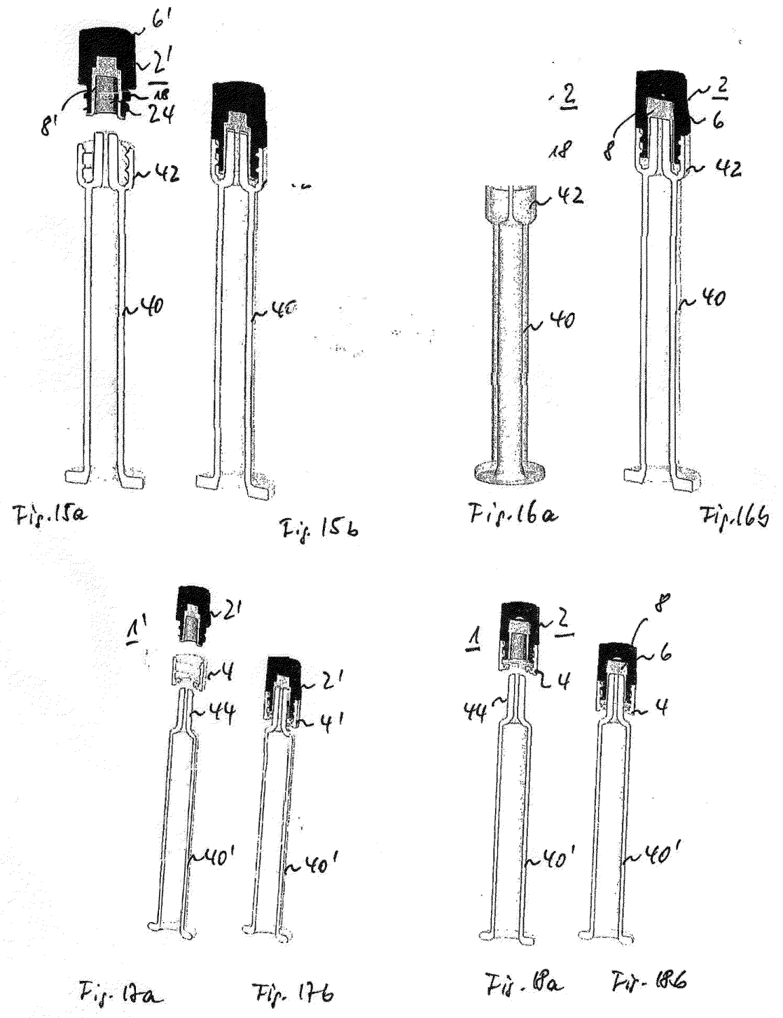

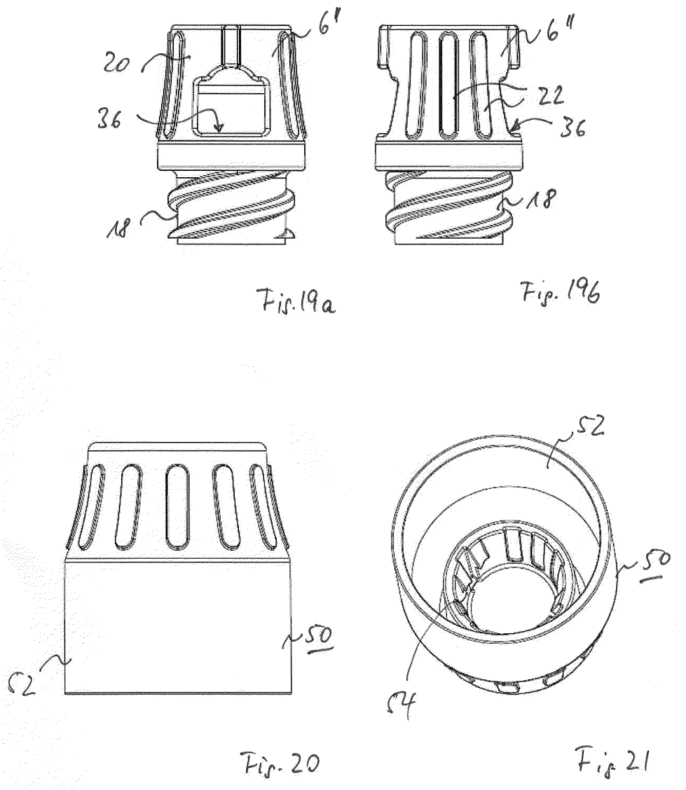

FIG. 2 a carrier cap of the closure cap of the closure system according to FIG. 1 in two different longitudinal sections,

FIG. 3 a sealing plug of the closure cap of the closure system according to FIG. 1 in longitudinal section,

FIG. 4 a threaded adapter piece 4 of the closure system according to FIG. 1,

FIG. 5 perspective section of the closure cap of the closure system as shown in FIG. 1,

FIG. 6 perspective view of the closure system according to FIG. 1 in assembled condition,

FIG. 7 the closure system according to FIG. 1 in assembled condition in two different longitudinal sections,

FIG. 8 an alternative closure system for a medicine container, in particular for a syringe prefilled with a medicament or medical active substance, in exploded view,

FIG. 9 the cap of the closure system as shown in FIG. 8 in longitudinal section in exploded view,

FIG. 10 perspective section of the closure cap of the closure system as shown in FIG. 8,

FIG. 11 exploded view of the closure system as shown in FIG. 8,

FIG. 12 perspective view of the closure system as shown in FIG. 8,

FIG. 13 perspective section of the closure system as shown in FIG. 8,

FIG. 14 the closure system as shown in FIG. 8 in longitudinal section,

FIG. 15 a syringe body closed with a closure cap as shown in FIG. 9 with an integrally formed thread system before (FIG. 15a) and after (FIG. 15b) attachment of the closure cap,

FIG. 16 a syringe body closed with a closure cap as shown in FIG. 5 with an integrally formed thread system before (FIG. 16a) and after (FIG. 16b) attachment of the closure cap,

FIG. 17 a syringe body closed with a closure system as shown in FIG. 8 before (FIG. 17a) and after (FIG. 17b) attachment of the closure system,

FIG. 18 a syringe body closed with a closure system as shown in FIG. 1 before (FIG. 18a) and after (FIG. 18b) attachment of the closure system

FIG. 19 an alternative embodiment of a carrier cap in two lateral views (FIG. 19a, 19b),

FIG. 20 an outer cap for use with the closure system according to FIG. 1 or FIG. 8 in side view,

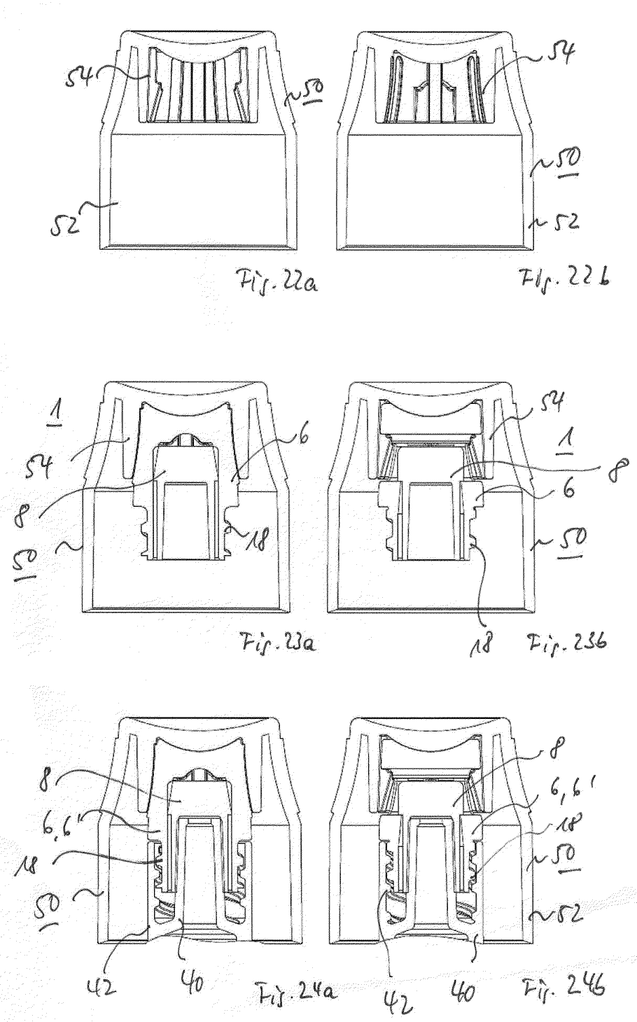

FIG. 21 the outer cap according to FIG. 20 in perspective view from below,

FIG. 22 the outer cap as shown in FIG. 20 in two variants, each in longitudinal section,

FIG. 23 a closure system with an outer cap according to FIG. 20 and a closure cap inserted therein in two variants, each in longitudinal section, and

FIG. 24 the locking system shown in FIG. 23 in mounted condition in two variants, each in longitudinal section.

Identical parts are marked with the same reference signs in all figures.

The closure system 1 shown in exploded view in FIG. 1 comprises as main components the closure cap 2, which is actually intended for closing a container prefilled with a medicament or medical active substance, in particular a syringe, and an associated threaded adapter piece 4. The closure cap 2 is in turn multi-component, in particular two-component, so that several actually competing design objectives can be achieved in the manner of a functional separation.

For this purpose, the closure cap 2 comprises as essential components, on the one hand, a rigid carrier cap 6 and, on the other hand, a sealing plug 8 made of a material that is softer and more malleable than the carrier cap 6, in particular made of rubber or TPE. The carrier cap 6, which is shown in FIG. 2 in two different longitudinal sections, is formed by a base body 10, which in turn is made of a plastic, in particular polypropylene (PP), a polyolefin, cyclo-olefin copolymer (COC), cyclo-olefin polymer (COP) or polycarbonate, in particular in the “medical grade” quality class. An inner channel 14, which extends in the longitudinal direction indicated by the longitudinal axis 12, is incorporated into this base body 10 and is intended to receive the sealing plug 8.

The base body 10 of the carrier cap 6 is provided with an external thread 18—preferably designed as a Luer thread, especially with regard to common connection systems in the field of medical equipment—in a “lower” contact area 16 intended for establishing the connection with the container or, in particular, the prefilled medical syringe.

In accordance with the design of the carrier cap 6, the closure cap 2 is thus intended for a screw connection with the medication container or the prefilled medical syringe. In order to facilitate particularly easy handling of the closure cap 2 when it is attached to the syringe body and also when it is later detached from the syringe body by screwing, i.e. by screwing on or off, the carrier cap 6 is provided with two longitudinally extending grip grooves 22 on its outer circumference in a handling area 20 that is offset from the contact area 16 in the longitudinal direction, as seen in the embodiment example. These form the gripping elements that make it particularly easy for the operator to apply force during the screwing movement.

The sealing plug 8 is shown in FIG. 3 in longitudinal section. Its outer contour is adapted to the inner channel 14 in the base body 10 of the carrier cap 6 in terms of dimensions and choice of geometry, so that it can be inserted into the latter. In its interior, on the other hand, it has a receiving channel 24 into which a corresponding orifice contour of the respective syringe or medical container can be inserted. Due to the choice of material for the sealing plug 8, namely rubber or TPE with correspondingly soft, elastic properties, the associated orifice area of the syringe or medical container can be sealed tightly.

The threaded adapter piece 4 is shown in FIG. 4 in longitudinal section (FIG. 4a) and in view from below (FIG. 4b). It is essentially manufactured in one piece from a plastic, in particular from polypropylene (PP), a polyolefin, cyclo-olefin copolymer (COC), cyclo-olefin polymer (COP) or polycarbonate, and comprises an outer casing 26, which is provided with an internal thread 28 adapted to the external thread 18 of the carrier cap 6. Furthermore, the threaded adapter piece 4 has a clamping ring 34 in an end region 30 on the base side, which can be pushed onto an end region of the syringe body and is provided with a number of circumferentially arranged clamping springs 32.

To form the closure cap 2, which is basically two-component in design, its components carrier cap 6 and sealing plug 8 are first joined together. In particular, these components should be pre-assembled for easier handling, so that only one component needs to be applied when the closure cap 2 is actually attached to the syringe body. In order to enable such a stable joining of the components, the inner channel 14 provided in the carrier cap 6 for receiving the sealing plug 8 is provided in the embodiment example with latching segments 36 formed on the inside, as can be seen in particular in the longitudinal sectional view in the appropriate direction according to FIG. 2a. Correspondingly, the sealing plug 8 has a peripheral latching edge 38 adapted to it on the outside. This is dimensioned and positioned to match the inner channel 14 in such a way that the sealing plug 8, after being inserted into the inner channel 14, is fixed in the inner channel of the carrier cap in a latching manner by interlocking the said latching elements.

The closure cap 2 resulting from this assembly is shown in perspective section in FIG. 5. In this form, this closure cap 2 is intended to be screwed directly onto the actual syringe body and is suitable if, according to its basic design, the latter is already equipped with a suitable thread system compatible with the external thread 18, for example in order to be able to screw a needle holder or the like onto the mouth area of the syringe body at a later stage.

Alternatively, the closure cap 2 can also be combined with the threaded adapter piece 4 to form the closure system 1, which can be slid onto syringe bodies with a tapered orifice area, for example. It is fixed to the syringe via the clamping ring 34, which can be pushed onto the end region of the syringe body and is provided with a number of circumferentially arranged clamping springs 32. For attachment to the syringe, the closure system 1 is preferably, and in an embodiment regarded as independently inventive, also pre-assembled here before actual attachment to the syringe by screwing the closure cap 2 with the external thread 18 into the internal thread 28 of the threaded adapter piece 4. A closure system 1 pre-assembled in this way is shown in perspective view in FIG. 6 and in two longitudinal sectional views in FIG. 7.

The closure cap 2 according to the invention provides, on the one hand, the desired high sealing effect due to the suitably designed sealing element 8 in this respect and, on the other hand, the desired high mechanical stability and load-bearing capacity due to the protective carrier cap 6.

FIG. 8 shows an exploded view of an alternative closure system 1′ with a closure cap 2′ in a design that is also regarded as independently inventive. It comprises as main components the alternative closure cap 2′ and also the associated threaded adapter piece 4. In this variant, the closure cap 2′ also has a two-component design, so that several actually competing design objectives can also be achieved in the manner of a functional separation. However, in this alternative embodiment, which is considered to be inventive in its own right, the closure cap 2′ is manufactured using a two-component injection molding process in which the two components are produced together from the outset. The components of the closure cap 2′ are shown in FIG. 9 in longitudinal section and in exploded view for the sake of clarity, although they are not shown separately for manufacturing reasons. FIG. 10, on the other hand, shows the complete closure cap 2′ produced using the two-component injection molding process in perspective section.

The closure cap 2′ as shown in FIG. 10 is also intended and suitable in this form for direct screwing onto the actual syringe body, provided that the latter is already equipped with a suitable thread system compatible with the external thread 18 according to its basic design, for example in order to be able to screw a needle holder or the like onto the mouth area of the syringe body at a later stage.

Alternatively, the closure cap 2′ can also be combined with the threaded adapter piece 4 to form the closure system 1, analogous to the variant described above, which can, for example, be pushed onto syringe bodies with a tapered mouth area. For attachment to the syringe, the closure system 1′ is preferably pre-assembled prior to actual attachment to the syringe by screwing the closure cap 2′ with the external thread 18 into the internal thread 28 of the threaded adapter piece 4, as indicated by the exploded view in FIG. 11. The closure system 1′ pre-assembled in this way is shown in perspective view in FIG. 12, in perspective section in FIG. 13 and in longitudinal section in FIG. 14.

FIGS. 15 to 18 each show a syringe body 40, 40′ in a version with an integrally formed thread system 42 (syringe body 40) or in a version without such a thread system and instead provided with a tapered mouth region 44 (syringe body 40′), namely in the illustration on the left in each case before and on the right after the attachment of the respective closure element.

The syringe body 40 provided with the molded-on thread system 42 can in particular be a prefillable, comparatively small-volume syringe body 40 (filling volume, for example 0.5 ml) with molded-on Luer thread, available as a standard product and widely used, for example as a “0.5 ml integrated polymer Luer thread syringe”. The thread system 42 can be provided, for example, in order to be able to screw a needle holder or the like onto the mouth area of the syringe body 40 at a later stage. The closure caps 2, 2′ described above with their preferred embodiments are basically intended to be screwed onto the actual syringe body 40 and are accordingly adapted to the thread system 42 in the design of their external thread 18. They can therefore be used directly with such a type of syringe body 40.

Alternatively, and with additional recourse to the threaded adapter piece 4, the closure caps 2, 2′ can also be used with the alternative type of syringe body 40′, also known as a “standard syringe”, with a tapered mouth region 44. Such a prefillable syringe body 40′ is widely used, for example, as a standard syringe with a filling volume of 1 ml. For this purpose, the respective closure system 1, 1′ can be pushed onto the mouth region 44 using the clamping ring 34 and clamped there.

An alternative design of a carrier cap 6″ is shown in FIG. 19 in two side views. In this design, a large number of grip grooves 22 running in the longitudinal direction are arranged on the outside of the handling area 20, which can significantly facilitate handling.

An outer cap 50 for use in the closure system 1, 1′ in addition to the closure cap 2, 2′ in an embodiment regarded as independently inventive is shown in lateral view in FIG. 20, in lateral perspective from below in FIG. 21 and in longitudinal section in FIG. 22. This has an outer casing 52, which is intended to completely enclose the closure cap 2, 2′ in the assembled state. Inside this outer casing 52 is a receiving basket 54 into which the closure cap 2, 2′ can be inserted or latched.

The receiving basket 54 is suitably adapted to the outer dimensions of the closure cap 2, 2′, particularly with regard to pre-assembly of the components, so that it can be inserted into the receiving basket 54 in a force-locking manner and fixed and guided by the latter. This becomes particularly clear in the illustration in FIG. 23, in each case in longitudinal section, in which the closure system 1, 1′ is shown with the outer cap 50 and the closure cap 2, 2′ inserted therein.

In the embodiment example, the outer cap 50 forms a tamper-evident closure for the closure cap 2,2′. For this purpose, the receiving basket 54 is provided with latching elements 56, which latch with the closure cap 2,2′ when it is inserted, so that removal is no longer possible. To gain access to the active substance, i.e. to “open” and prepare the syringe closed in this way, a lid area of the outer cap 50 can then be torn off by means of suitable predetermined breaking points in the outer cap 50, for example, so that the closure cap 2,2′ is exposed and thus made accessible.

The pre-assembled ensemble of outer cap 50 and inner closure cap 2,2′ shown in FIG. 23 can then be attached to the respective syringe particularly easily, especially by screwing it on. This is shown in longitudinal section in FIG. 24. In particular, the thread 18 is in screw connection with the internal thread system 42 of the syringe body 40. The outer cap 50 is adapted to the outer diameter of the syringe by a suitable choice of geometry and size of its outer casing 52, which is also regarded as an independently inventive embodiment.

### LIST OF REFERENCE SYMBOLS

1, 1′ closure system
2, 2′ closure cap
4 threaded adapter piece
6, 6′ carrier cap
8,8′ sealing plug
10 base body
12 longitudinal axis
14 inner channel
16 contact region
18 external thread
20 handling region
22 grip grooves
24 receiving channel
26 outer casing
28 internal thread
30 end region
32 clamping springs
34 clamping ring
36 latching segments
38 latching edge
40, 40′ syringe body
42 thread system
44 mouth region
50 outer cap
52 outer casing
54 receiving basket
56 latching elements

1. Closure system for a syringe body prefilled with a medicament or medical active substance, with a closure cap having a rigid carrier cap which is provided with an external thread in a contact region and has an inner channel which extends in a longitudinal direction and in which a sealing plug made of a material which is softer and more deformable than the carrier cap and preferably made of rubber or TPE is arranged, wherein the inner channel provided in the carrier cap is provided with latching segments arranged on its inner circumference, and wherein the sealing plug has a latching edge adapted thereto and extending around the outside and dimensioned such that the sealing plug is fixed in a latching manner after being inserted into the inner channel and with an outer cap surrounding the closure cap, which has a receiving basket for the closure cap arranged at the end inside an outer casing.

2. Closure system according to claim 1, in which the carrier cap of the closure cap is made of a plastic, preferably of polypropylene (PP), a polyolefin, cyclo-olefin copolymer (COC), cyclo-olefin polymer (COP) or polycarbonate.

3. Closure system according to claim 1, in which the carrier cap of the closure cap is provided on its outer circumference with a number of, preferably two, longitudinally extending grip grooves in a handling region which is arranged offset from the contact region as viewed in the longitudinal direction.

4. Closure system according to claim 1, where the external thread of the closure cap is designed as a Luer thread.

5. Closure system according to claim 1, to 4, where the closure cap is obtained by production using a two-component injection molding process.

6. Closure system according to claim 1, with a threaded adapter piece which is provided in an outer casing with an internal thread adapted to the external thread of the carrier cap, and which has a clamping ring which can be pushed onto a tapering mouth region of the syringe body and is provided with a number of circumferentially arranged clamping springs.

7. Closure system according to claim 6, the threaded adapter piece of which is made of a plastic, preferably of polypropylene (PP), a polyolefin, cyclo-olefin copolymer (COC), cyclo-olefin polymer (COP) or polycarbonate.

8. Closure system according to claim 1, the outer cap of which forms a tamper-evident closure for the closure cap.
